(19) United States
(12) Patent Application Publication
(10) Pub. No.: US 2004/0224000 A1
(43) Pub. Date: Nov. 11, 2004

(54) Implants for non-radioactive brachytherapy of hormonal-insensitive cancers

(76) Inventor: Romano Deghenghi, St. Cergue (CH)

Correspondence Address: WINSTON & STRAWN, PATENT DEPARTMENT, 1400 L STREET, N.W., WASHINGTON, DC, 20005-3502, US

(21) Appl. No.: 10/430,132

(22) Filed: May 5, 2003

(51) Int. Cl. A61K 38/22; A61K 9/14

(52) U.S. Cl. 424/423; 514/12

(57) ABSTRACT

Implants are described for use in a novel therapy of hormone-insensitive tumors. The implants are inserted near, around or inside such tumors to provide a high local concentration and sustained release of a gonadotrophin-release hormone agonist or antagonist and a direct inhibitory action on the growth of such tumors. As the implants are not radioactive, the deleterious side-effects of radioactive treatments are avoided.

### BACKGROUND

Hormone-dependent cancers, such as prostate and breast cancers, are presently treated with GnRH (Gonadotrophin Releasing Hormone, which is also known as LHRH, Luteinizing Hormone Releasing Hormone) agonists or antagonists. Their action is at the pituitary-gonadal axis, where they suppress the production of gonadotrophins (i.e., LH and FSH) which are responsible for the synthesis of androgens (testosterone) in the testes or of estrogens (estradiol) in the ovaries. These hormones are known to stimulate the growth of certain cancers in men and women. This inhibition results in a biochemical castration with a resulting effective control of the growth of the androgen-dependent (prostate) or estrogen-dependent (breast) cancers.

GnRH analogs are conveniently administered subcutaneously or intramuscularly in form of depot formulations. Such formulations are generally in form of rods (e.g., goserelin, Zoladex®), suspensions of microspheres or microparticles (e.g., leuprolide, tryptorelin) or in a gel form (Leuprogel®). These formulations provide a sustained release of the active principle for one to four months or longer. The clinical action of these analogs is unfortunately of a palliative nature and will cease to be effective when the cancer inexorably progresses from a hormone-dependent to a hormone-independent form.

The reasons for this progression are poorly understood (cf. P. Hàrkonen et al. J. Clin. End. Met. 88, 705-712, 2003). Some reasons that have been suggested include variation in 17-hydroxysteroid-dehydrogenase activity in mutated cancer cells or a presence of EGF (epidermal growth factor) or IGF (insulin growth factor) both of which are known to stimulate the proliferation of prostate cancers. An optional therapy to improve survival is provided by administering radioactive brachytherapy (short-spaced therapy). This therapy includes the implanting, near or inside the cancerous tissue, of varying doses of a gamma-emitting radioisotope such as, e.g., 125-Iodine (cf. A. C. Pellizzon et al., Urol. Int. 70, 200-204, 2003; P. G. Koutrouvelis et al., J. Urol., 169, 1331-1336, 2003).

Several techniques are well known and available to insert the radioactive isotope near, around or inside the cancerous tissue. In the case of the prostate, the insertion of needles or seeds of radioactive material is known as “3-dimensional computerized tomography guided pararectal brachytherapy” (cf. P. G. Koutrouvelis et al. loc. cit.). Another technique is known as “intraoperative computer-optimized transperineal ultrasound guided prostate brachytherapy” (cf. M. J. Zelefsky et al., Int. J. Radiat. Oncol. Biol. Phys., 55, 956-963, 2003). Despite these techniques, the side-effects of brachytherapy, particularly in the prostate, are severe and vary from dysuria to incontinence to radiation damage affecting normal neighboring organs and tissues such as the bladder, rectum and others.

Thus, there is a need for alternative treatments for these cancerous tumors that eliminates or reduces such undesirable side-effects. The present invention now provides both a product and method that satisfy this need.

### SUMMARY OF THE INVENTION

The invention relates a method for treating a hormonal-independent tumor in a mammal by the administration of an implant which releases a high therapeutic concentration of an effective GnRH analog, directly in or adjacent the tumor wherein the GnRH is released in an amount effective to inhibit the growth of the tumor. The GnRH analog may be a GnRH agonist, such as buserelin, tryptorelin, goserelin, avorelin, deslorerin, or leuprolide, or an antagonist of LHRH, such as teverelix, cetrorelix, ganirelix, or abarelix.

Another embodiment of the invention relates to a brachytherapy implant which comprises a therapeutically effective amount of a GnRH analog for placement directly in or adjacent the tumor wherein the GnRH is released in an amount effective to inhibit the growth of the tumor.

In a further embodiment, the invention relates to a method of avoiding radiation exposure in a mammal receiving brachytherapy for treatment of a hormone-independent tumor which comprises formulating a non-radioactive brachytherapy implant which comprises a therapeutically effective amount of a GnRH analog for placement directly in or adjacent the tumor wherein the GnRH is released in an amount effective to inhibit the growth of the tumor.

Yet another embodiment relates to a method for forming a brachytherapy implant which comprises incorporating into the implant a GnRH analog in a therapeutically effective amount such that the implant can be placed directly in or adjacent the tumor so that the GnRH is released in an amount effective to inhibit the growth of the tumor.

In addition, the invention relates to the use of a composition comprising a biodegradable polymer and a GnRH analog for preparing a brachytherapy implant. The implant is preferably inserted directly into the tumor to achieve interstitial brachytherapy. When the implant is to be inserted into the prostate, for example, computer-optimized transperineal ultrasound guided prostate brachytherapy can be used.

The implant preferably provides an extended release of the GnRH over a time of at least one to four months, and is used to treat tumors present in the prostate or breast of the mammal. The GnRH may be provided in a composition in association with a biodegradable material applied to a support as a coating, wherein the coated support is the implant. The implant may also be in the form of a rod, microparticles or microspheres, a biocompatible gel, or slow-release microcrystals. If desired, the implant can include a suitable radio-opaque agent to improve visualization when administering the implant.

### DETAILED DESCRIPTION OF THE PREFERRED EMBODIMENTS

As used herein, the expression “mammal” refers to any mammalian subject, such as mice, rats, guinea pigs, cats, dogs, human beings, cows, horses, sheep, or other livestock.

“Cancer” comprises tissue that grows by either increased cellular proliferation and/or decreased apoptosis.

As used herein, the term “treating” includes inhibiting the disease, disorder or condition, i.e., arresting its development, or relieving the disease, disorder or condition, i.e., causing regression of the disease, disorder and/or condition.

“Intratumoral” administration means implanting a reservoir of a therapeutic agent(s) inside a tumor. Intratumoral administration is advantageous for tumor treatment because the outer cell layers of tumors are often composed of a high percentage of necrotic cells and/or connective and support tissue which slow and/or impede the extra-tumoral vascular or parenteral delivery of therapeutic agents to the actively growing cancer cells at the center of solid tumors.

“Biodegradable” means capable of being biologically decomposed. A “biodegradable” polymer can be biologically decomposed into units which may be either removed from the biological system and/or chemically incorporated into the biological system.

“Solid tumor” means a locus of tumor cells where the majority of the cells are tumor cells or tumor-associated cells.

The expression “extended release”, as used herein, includes various forms of release of a therapeutic agent over time, such as by controlled release, timed release, sustained release, delayed release, long acting, pulsatile delivery, or immediate release that occurs with various rates. The ability to obtain extended release is well-known to the skilled artisan. When used in accordance with the method of the invention, the implants provide extended release of the GnRH analog into the solid tumor of a subject having one or more of such tumors, preferably for a period of from about one month to four months, although for certain treatments, the release profile can extends over a longer time for example, to up to 6 months or even one year.

The treatment method includes the use of implant, preferably one that is biodegradable, for treating a subject having a solid tumor. Moreover, the tumor treated in the invention can be either primary or a secondary tumor resulting from metastasis of cancer cells elsewhere in the body to the chest. Preferably, the tumor is one which is hormone non-responsive, i.e., the hormones of the subject or patient do not significantly contribute to further growth of the tumor. This is a problematic condition since the use of hormone treatments are not recognized as being of significance in treating the tumor. This condition often occurs in prostate or breast tumors.

The scientific field does not have a uniform view regarding the effect of hormone affecting therapeutics for treating hormone-independent tumors or cancers, but it appears that there is a recognition of benefits for such treatments (see. e.g., M. Marelli et al., Endocrinology, 1999 January; 140 (1): 329-34; D. Dondi et al., Int. J. Cancer, 1998, May 18; 76 (4): 506-11; M. Montagnoni Marelli et al., Arch Ital Urol Androl, 1997 September; 69 (4) 257-63; and R. M. Moretti et al., J Clin Endocroinol Metab, 1996, November; 81 (11): 3930-7). The present invention furthers these treatments by administering the hormone-affecting agent, in this case a GnRH analog, directly into or immediately adjacent the tumor itself so that the optimum therapeutic effect can be obtained. The logic behind this procedure is the same as that used in brachytherapy, except in the present invention, a non-radioactive, biodegradable device is implanted to achieve therapeutic results. These devices will be referred to as “implants” herein.

The implants generally include a biodegradable polymer or composition which is used alone or in combination with other biocompatible extended release of the GnRH analog into the solid tumor of a subject having one or more of such tumors, preferably for a period of from about one month to four months, although for certain treatments, the release profile can extends over a longer time for example, to up to 6 months or even one year.

The treatment method includes the use of implant, preferably one that is biodegradable, for treating a subject having a solid tumor. Moreover, the tumor treated in the invention can be either primary or a secondary tumor resulting from metastasis of cancer cells elsewhere in the body to the chest. Preferably, the tumor is one which is hormone non-responsive, i.e., the hormones of the subject or patient do not significantly contribute to further growth of the tumor. This is a problematic condition since the use of hormone treatments are not recognized as being of significance in treating the tumor. This condition often occurs in prostate or breast tumors.

The scientific field does not have a uniform view regarding the effect of hormone affecting therapeutics for treating hormone-independent tumors or cancers, but it appears that there is a recognition of benefits for such treatments (see. e.g., M. Marelli et al., Endocrinology, 1999 January; 140 (1): 329-34; D. Dondi et al., Int. J. Cancer, 1998, May 18; 76 (4): 506-11; M. Montagnoni Marelli et al., Arch Ital Urol Androl, 1997 September; 69 (4) 257-63; and R. M. Moretti et al., J CLin Endocroinol Metab, 1996, November; 81 (11): 3930-7). The present invention furthers these treatments by administering the hormone-affecting agent, in this case a GnRH analog, directly into or immediately adjacent the tumor itself so that the optimum therapeutic effect can be obtained. The logic behind this procedure is the same as that used in brachytherapy, except in the present invention, a non-radioactive, biodegradable device is implanted to achieve therapeutic results. These devices will be referred to as “implants” herein.

The implants generally include a biodegradable polymer or composition which is used alone or in combination with other biocompatible polymers or copolymers, so long as the additional polymers or copolymers do not interfere undesirably with the biodegradable characteristics of the composition. Preferably, biodegradable polymers of the present invention comprise more than about 50% of the implant. Blends of the polymers may offer even greater flexibility in designing the precise release profile desired for targeted drug delivery or the precise rate of biodegradability desired. Examples of biocompatible or biodegradable polymers include poly(phosphoesters), poly(esters), poly(lactides), poly(glycolides), poly(caprolactones), poly(anhydrides), poly(amides), poly(urethanes), poly(esteramides), poly(orthoesters), poly(dioxanones), poly(acetals), poly(ketals), poly(carbonates), poly(imino-carbonates), poly(orthocarbonates), poly(phosphazenes), poly(hydroxybutyrates), poly(hydroxyvalerates), poly(alkylene oxalates), poly(alkylene succinates), poly(malic acids), poly(amino acids), poly(vinylpyrrolidone), poly(ethylene glycol), poly(hydroxycellulose), chitin, chitosan, and copolymers, terpolymers, or combinations or mixtures of the above materials.

Pharmaceutically acceptable polymeric carriers may also be included and these can vary over a wide range of materials. Without limitation, such materials may include well-known diluents, binders and adhesives, lubricants, disintegrants, colorants, bulking agents, flavorings, sweeteners, and miscellaneous materials such as buffers and adsorbents, in order to prepare a particular medicated composition. The addition of such materials is limited to those additional materials which will not interfere with the biocompatibility, biodegradability and physical state desired of the implants of the invention.

For delivery, the GnRH analog is added to the polymer composition. The agent or substance is either dissolved to form a homogeneous solution of reasonably constant concentration in the polymer composition, or dispersed to form a suspension or dispersion within the polymer composition at a desired level of “loading” (grams of biologically active substance per grams of total composition including the biologically active substance, usually expressed as a percentage).

While it is possible that the biodegradable polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the invention that, in a preferred embodiment, no solvent is needed to form the desired composition.

The polymer composition of the invention may be a rigid solid article, a flexible solid article or material, or a flowable material. By “flowable” is meant the ability to assume, over time, the shape of the space containing it at body temperature. This includes, for example, gel compositions or even liquid compositions that are capable of being delivered into, upon or adjacent the tumor. For certain treatments, a manually operated syringe fitted with, for example, a 23-gauge needle can be used, or these compositions can be delivered through a catheter.

The term “flowable” includes highly viscous materials that are “gel-like” at room temperature. These may be delivered to the desired site in the tumor by pouring, squeezing from a tube, or being injected with any one of the commercially available power injection devices that provide injection pressures greater than would be exerted by manual means alone for highly viscous, but still flowable, materials. Such flowable polymer compositions have the advantage of providing controllable and effective release of the GnRH analog over time.

When the polymer used is itself flowable, the polymer composition of the invention, even when viscous, need not include a biocompatible solvent to be flowable, although trace or residual amounts of biocompatible solvents may still be present. The degree of viscosity of the polymer can be adjusted by the molecular weight of the polymer, as well as by mixing any cis- and trans-isomers of the diol in the backbone of the polymer.

The polymer composition of the invention can be administered by a variety of routes. For example, if flowable, it can be injected directly into the solid tumor being treated with a needle, such as a Turner Biopsy Needle or a Chiba Biopsy Needle.

In its simplest form, the implant is a simple solution or dispersion of the GnRH analog in a polymer matrix having an unstable (biodegradable) bond incorporated into the polymer backbone. In a particularly preferred embodiment, a solid article comprising the composition of the invention is inserted into the solid tumor being treated by implantation, injection, or otherwise being placed within the tumor of the subject being treated, for example, during or after the surgical removal of a portion of visibly cancerous tissue.

The GnRH analog of the composition and the polymer may form a homogeneous matrix, for example in the form of microspheres, or the antineoplastic agent may be encapsulated in some other way within the polymer. For example, the antineoplastic agent may be first encapsulated in a microsphere and then combined with the polymer in such a way that at least a portion of the microsphere structure is maintained. Alternatively, the antineoplastic agent may be sufficiently immiscible in the polymer of the invention that it is dispersed as small droplets, rather than being dissolved, in the polymer.

As a structural medical device, the polymer compositions of the inventions provide a wide variety of physical forms having specific chemical, physical and mechanical properties suitable for insertion into the tumor being treated, in addition to being a composition that degrades in vivo into non-toxic residues. Specifically, the composition itself may be fabricated to take the shape of a rod, needle or pin that can be manually or automatically inserted into the tumor mass.

The implants can be prepared in several ways. The polymer can be melt processed using conventional extrusion or injection molding techniques, or these products can be prepared by dissolving in an appropriate solvent, followed by formation of the device, and subsequent removal of the solvent by evaporation or extraction, e.g., by spray drying. By these methods, the polymers may be formed into articles of almost any size or shape desired, for example, implantable or injectable needles, rods, microspheres, or other microparticles. Typical medical articles also include coatings to be placed on other implant devices.

Additional implants for use in the invention include those disclosed in U.S. Pat. Nos. 6,159,490, 6,077,523 and 5,945,128, as well as in PCT publications WO/03/022297, WO02/30393 and WO01/54662. In these patents and patent applications, the active ingredient is a GnRH analog as described herein, and the implant is sized appropriately to be administered through a needle or brachytherapy system.

Once inserted, the polymer composition of the invention should preferably remain in at least partial contact with tumor or the cancerous cells thereof. The implanted or injected composition will release the GnRH analog contained within its matrix within, upon or next to the tumor at a controlled rate until the substance is depleted, following the general rules for diffusion or dissolution from a rigid, flexible or flowable biodegradable polymeric matrix.

The method of the invention can be used to treat a solid tumor in a mammal by the intratumoral administration of an implant comprising a biodegradable polymer; and at least one GnRH analog in an amount effective to inhibit the growth of the tumor when administered by intratumoral injection. As noted above, the implant is administered to tumor that is no longer hormone dependent.

The invention preferably relates to the insertion near, around or inside a prostate or breast tumor, which is or will become hormone-independent, of a non-radioactive device by brachytherapy procedures. The most preferred devices are one or more of the following: a sustained release formulation of an effective GnRH analog to provide a local concentration of such GnRH analog at a local concentration several order of magnitude that which is usually achieved when such device is placed subcutaneously or intramuscularly elsewhere. As noted above, these formulations may be in the form of a rod, in microparticulate form in a suspension fluid, in a sustained release gel formulation, or in a sustained release microcrystalline form.

If needed, such devices can be visualized with ultrasound techniques or could be optionally coated with radio-opaque agents such as iodine, barium salts, or metals such as tantalum, tungsten and the like. Such devices can be placed into the patient at intervals of several months depending on the duration of the desired remission. Being non-radioactive, they do not exhibit the side-effects noted with conventional brachytherapy.

While the different types of formulations are generally known, these have in the past been primarily administered by way of subcutaneous or intramuscular injection. The use of sustained release implants of a LHRH (GnRH) analog directly around or inside a hormone-independent cancer tissue, such as the breast or prostate now leads to a highly effective new treatment for these debilitating diseases.

What is claimed is:

1. A method for treating a hormonal-independent tumor in a mammal by the administration of an implant which releases a high therapeutic concentration of an effective GnRH analog, directly in or adjacent the tumor wherein the GnRH is released in an amount effective to inhibit the growth of the tumor.

2. The method of claim 1 wherein the implant provides an extended release of the GnRH over a time of at least one to four months.

3. The method of claim 1 wherein the tumor is present in the prostate of the mammal.

4. The method of claim 1 wherein the tumor is present in the breast of the mammal.

5. The method of claim 1 wherein the implant is in the form of a rod.

6. The method of claim 1 wherein the implant is in the form of microparticles or microspheres.

7. The method of claim 1 wherein the implant is in form of a biocompatible gel.

8. The method of claim 1 wherein the implant is in form of slow-release microcrystals.

9. The method of claim 1 wherein the implant includes a suitable radio-opaque agent to improve visualization when administering the implant.

10. The method of claim 1 wherein the GnRH is provided in a composition in association with a biodegradable material.

11. The method of claim 10 wherein the composition is applied to a support as a coating, wherein the coated support is the implant.

12. The method of claim 1 wherein the GnRH analog is a GnRH agonist.

13. The method of claim 12 wherein the GnRH agonist is buserelin, tryptorelin, goserelin, avorelin, deslorerin, or leuprolide.

14. The method of claim 1 wherein the GnRH analog is an antagonist of LHRH.

15. The method of claim 14 wherein the GnRH antagonist is teverelix, cetrorelix, ganirelix, or abarelix.

16. The method of claim 1 wherein the implant is inserted in the tumor to achieve interstitial brachytherapy.

17. The method of claim 1 wherein the implant is inserted into the prostate with computer-optimized transperineal ultrasound guided prostate brachytherapy.

18. A brachytherapy implant which comprises a therapeutically effective amount of a GnRH analog for placement directly in or adjacent the tumor wherein the GnRH is released in an amount effective to inhibit the growth of the tumor.

19. The brachytherapy implant of claim 18 in the form of a rod.

20. The brachytherapy implant of claim 18 in the form of microparticles or microspheres.

21. The brachytherapy implant of claim 18 in form of a biocompatible gel.

22. The brachytherapy implant of claim 18 in form of slow-release microcrystals.

23. The brachytherapy implant of claim 18 which includes a suitable radio-opaque agent to improve visualization when administering the implant.

24. The brachytherapy implant of claim 18 wherein the GnRH is provided in a composition in association with a biodegradable material.

25. The brachytherapy implant of claim 24 wherein the composition is applied to a support as a coating, wherein the coated support is the implant.

26. The brachytherapy implant of claim 18 wherein the GnRH analog is a GnRH agonist.

27. The brachytherapy implant of claim 26 wherein the GnRH agonist is buserelin, tryptorelin, goserelin, avorelin, deslorerin, or leuprolide.

28. The brachytherapy implant of claim 18 wherein the GnRH analog is an antagonist of LHRH.

29. The brachytherapy implant of claim 28 wherein the GnRH antagonist is teverelix, cetrorelix, ganirelix, or abarelix.

30. A method of avoiding radiation exposure in a mammal receiving brachytherapy for treatment of a hormone-independent tumor which comprises formulating a non-radioactive brachytherapy implant which comprises a therapeutically effective amount of a GnRH analog for placement directly in or adjacent the tumor wherein the GnRH is released in an amount effective to inhibit the growth of the tumor.

31. The method of claim 30 wherein the implant is in the form of a rod.

32. The method of claim 30 wherein the implant is in the form of microparticles or microspheres.

33. The method of claim 30 wherein the implant is in form of a biocompatible gel.

34. The method of claim 30 wherein the implant is in form of slow-release microcrystals.

35. The method of claim 30 wherein the implant includes a suitable radio-opaque agent to improve visualization when administering the implant.

36. The method of claim 30 wherein the GnRH is provided in a composition in association with a biodegradable material.

37. The method of claim 36 wherein the composition is applied to a support as a coating, wherein the coated support is the implant.

38. The method of claim 30 wherein the GnRH analog is a GnRH agonist.

39. The method of claim 38 wherein the GnRH agonist is buserelin, tryptorelin, goserelin, avorelin, deslorerin, or leuprolide.

40. The method of claim 30 wherein the GnRH analog is an antagonist of LHRH.

41. The method of claim 40 wherein the GnRH antagonist is teverelix, cetrorelix, ganirelix, or abarelix.

42. A method for forming a brachytherapy implant which comprises incorporating into the implant a GnRH analog in a therapeutically effective amount such that the implant can be placed directly in or adjacent the tumor so that the GnRH is released in an amount effective to inhibit the growth of the tumor.

43. The method of claim 42 wherein the GnRH is provided in a composition in association with a biodegradable material.

44. The method of claim 43 wherein the composition is applied to a support as a coating, wherein the coated support is the implant.

45. The method of claim 42 wherein the wherein the implant is in the form of a rod.

46. The method of claim 42 wherein the implant is in the form of microparticles or microspheres.

47. The method of claim 42 wherein the implant is in form of a biocompatible gel.

48. The method of claim 42 wherein the implant is in form of slow-release microcrystals.

49. The method of claim 42 wherein the implant includes a suitable radio-opaque agent to improve visualization when administering the implant.

50. Use of a composition comprising a biodegradable polymer and a GnRH analog for preparing a brachytherapy implant.

51. Use according to claim 50 wherein the implant is in the form of a rod.

52. Use according to claim 50 wherein the implant is in the form of microparticles or microspheres.

53. Use according to claim 50 wherein the implant is in form of a biocompatible gel.

54. Use according to claim 50 wherein the implant is in form of slow-release microcrystals.

55. Use according to claim 50 wherein the implant includes a suitable radio-opaque agent to improve visualization when administering the implant.

56. Use according to claim 50 wherein the GnRH is provided in a composition in association with a biodegradable material.

57. Use according to claim 56 wherein the composition is applied to a support as a coating, wherein the coated support is the implant.

58. Use according to claim 50 wherein the GnRH analog is a GnRH agonist.

59. Use according to claim 58 wherein the GnRH agonist is buserelin, tryptorelin, goserelin, avorelin, deslorerin, or leuprolide.

60. Use according to claim 50 wherein the GnRH analog is an antagonist of LHRH.

61. Use according to claim 60 wherein the GnRH antagonist is teverelix, cetrorelix, ganirelix, or abarelix.

62. The brachytherapy implant prepared by claim 50.
